(19) United States
(12) Patent Application Publication
(10) Pub. No.: US 2017/0265957 A1
(43) Pub. Date: Sep. 21, 2017

(54) SURGICAL BIB, METHOD, AND KIT

(71) Applicant: Medline Industries, Inc., Mundelein, IL (US)

(72) Inventors: Mark Spencer Chua, Northbrook, IL (US); David Preskill, Highland Park, IL (US)

(21) Appl. No.: 15/071,310

(22) Filed: Mar. 16, 2016

(51) Int. Cl. A61B 46/20; A61B 17/42

(52) U.S. Cl. CPC A61B 46/20; A61B 17/42; A61B 2046/205

(57) ABSTRACT

Disclosed are a surgical bib and kit useful in assisting a surgeon in locating and orienting the situs of a surgical incision. Generally, the surgical drape kit includes a fenestrated surgical drape and a fenestrated surgical bib sized to cover the drape fenestration and having a bib fenestration formed therein to permit surgical access therethrough. The surgical bib includes at least one surgical guide marking that may be used to position the surgical bib on the patient with reference to one or more anatomic features of the patient.

### TECHNICAL FIELD

This disclosure relates to a device for assisting a surgeon in making a surgical incision.

### BACKGROUND OF THE INVENTION

Currently, a traditional cesarean section procedure, also known as a “C-section,” is most commonly performed with the patient lying recumbent on an operating table. Generally, surgical patients are covered with a sterile surgical drape to isolate the area of the patient on which the surgery will be performed from the rest of the patient so that a sterile field can be created and maintained. In the case of a C-section, the area isolated by the drape is the patient's abdomen. A surgeon makes an incision in the abdomen in this isolated region.

C-section procedures necessarily leave the patient with a sizeable scar that may later be visible when the patient wears certain attire. For cosmetic purposes, it is desirable that the scar should be neat, bilaterally symmetric with respect to the centerline of the patient, and oriented generally perpendicular with regard to this centerline. With many conventional drapes, it can be difficult to achieve this result.

The portion of the surgical drape that overlays the patient's abdomen typically includes a fenestration that provides surgical access to the patient's abdomen. C-section procedures generate substantial amounts of bodily fluids, and it is necessary to mitigate the flow of these fluids to undesirable areas. To address this concern, it is known to fill the fenestration of the drape with an adhesive film known in the field as “incise film” and to provide a fluid collection pouch proximal the fenestration. The incise film covers the fenestration in the surgical drape, and is constructed of a material through which an incision in the patient's skin may be made. Via adhesive attachment to the patient, the incise film inhibits fluids generated during the surgical procedure from escaping beneath the drape. Fluids instead flow over the drape into the collection pouch.

The incise film is typically a flat film and it can be difficult to adhere the film to the patient's anatomy, which is very rounded. Also, the adhesive connection of the incise film to the underlying skin will often cause the underlying skin to bunch up or distort. This can result in an incision that is irregular in shape, despite the best efforts of the surgeon, and consequently may lead to a jagged scar. Also, fragments of the incise film may detach while the surgeon is making the incision and those small fragments may migrate into the incision, increasing the chances of an infection or otherwise inhibiting proper healing. Some surgeons do not prefer the tactile feel of cutting through incise film when making an incision.

In addition, with some known drapes, once the drape is placed over the patient and the area in which the surgery is to be performed is isolated, the surrounding anatomy of the patient is occluded by the opaque drape material. This can make it difficult for a surgeon to precisely locate and orient the incision. This configuration can lead the surgeon to make an incision that is not bilaterally symmetric or that is skewed relative to the patient's centerline.

A new device, namely, a surgical bib has now been devised, and a kit that includes the bib and a corresponding surgical drape are likewise disclosed herein. The flexible bib sheet is made of a flexible sheet of material having a patient-facing side and a surgeon-facing side opposite the patient-facing side. Generally, the surgical drape kit includes a fenestrated surgical drape and this flexible bib sheet. A bib fenestration is formed in the flexible bib sheet thereby permitting surgical access to a patient through the bib fenestration. The flexible bib sheet includes at least one surgical guide marking that is visible on the surgeon-facing side of the flexible bib sheet. The flexible bib sheet is sized such that it at least partially covers the fenestration in the surgical drape, and preferably substantially or completely covers the fenestration in the surgical drape when properly positioned in intended use.

At least a portion of the patient-facing side of the flexible bib sheet is covered with an adhesive field, either upon manufacture or by contemporaneous application of adhesive by the surgeon, or in some instances via application of supplemental adhesive to a bib that already includes a coating of adhesive. The adhesive field comprises an adhesive suitable for adhering a portion of the patient-facing side of the flexible bib to the patient's skin in an area surrounding the surgical site. The adhesive field of the surgical bib sheet may be covered with a removable film that may be removed to expose the adhesive at a time when the surgical bib is to be applied to the patient.

Although it is contemplated that the bib could be used with a full incise film drape, or with a drape having no incise film, more preferably the surgical drape is provided with a drape window that is only partially covered by incise film. This incise film includes an open fenestration intended to be placed proximal the portion of the patient's body where the incision is to be made, this open fenestration in the incise film defining the drape fenestration. The bib is used to assist the surgeon in more precisely locating and orienting the situs of the incision. When the surgical drape is placed over a patient, the flexible bib sheet is placed over the patient and positioned over the patient's body by aligning at least one surgical guide marking on the flexible bib sheet with at least one anatomic feature of the patient and by locating the surgical bib fenestration over the area of the patient in which an incision will be made. The bib may include a rule or scale to assist the surgeon in making a bilaterally symmetric incision.

The bib and drape may be provided together as a kit. The kit optionally may also include a container of adhesive, which may be used to provide supplemental adhesive or may be used as the primary source of adhesive if the bib is manufactured without its own coating of adhesive.

### BRIEF DESCRIPTION OF THE DRAWINGS

FIG. 1 is a perspective view of a surgical drape of the surgical drape kit positioned over a pregnant patient in need of a C-section procedure, with an anesthesia screen obscuring the patient's view of the surgical area.

FIG. 2 is a top plan view of the surgical drape shown in FIG. 1.

FIG. 3 is a top plan view, enlarged with respect to FIG. 2, of the fenestration of the surgical drape shown in FIG. 2.

FIG. 4 is a top plan view similar to FIG. 3 but illustrating the drape after removal of the backing material that originally is disposed over the incise film surrounding the drape fenestration.

FIG. 5 is a localized top view of the drape shown in FIG. 2, shown positioned over the abdomen of a patient.

FIG. 6 is a perspective view of a flexible bib sheet useful in conjunction with the surgical drape depicted in the previous figures, shown partially separated from a removable backing.

FIG. 7 is a top plan view of the bib shown in FIG. 6.

FIG. 8 is a top view of the bib and drape depicted above, shown with the backing of the bib removed and showing the bib placed over the surgical drape when the drape in the position shown in FIG. 5.

FIG. 9 is an idealized perspective view of a packaged surgical drape kit containing the surgical drape and flexible bib sheet depicted above and an optional container of adhesive.

### DETAILED DESCRIPTION

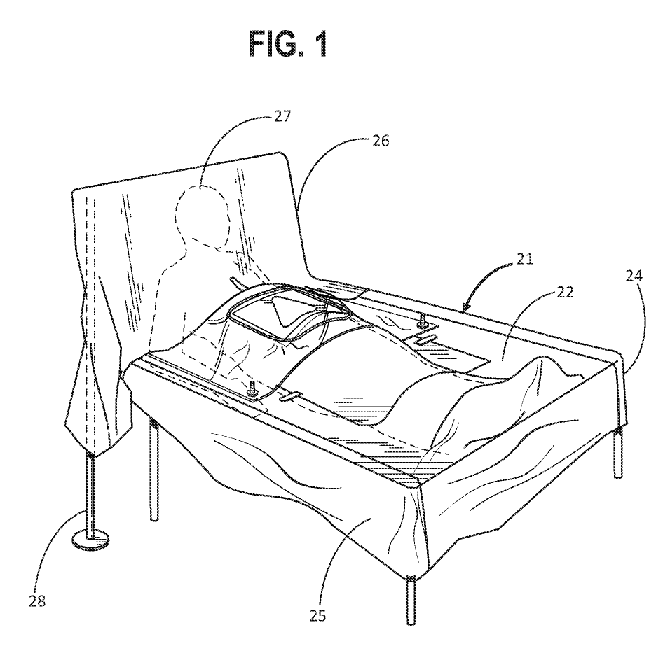

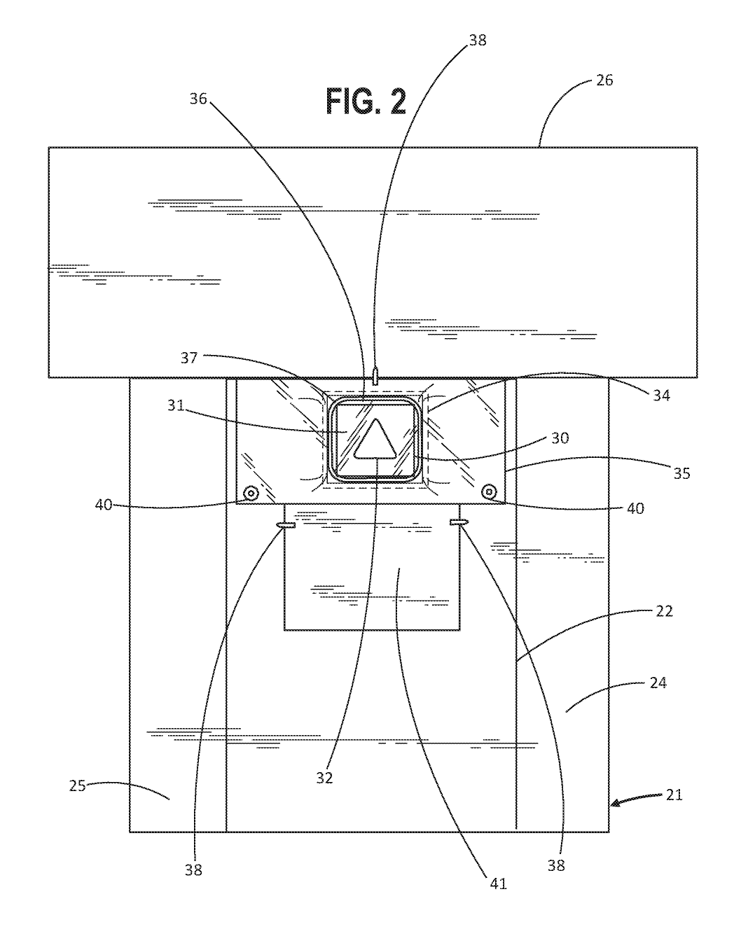

Referring to FIG. 1, the surgical drape 21 has a main sheet 22, a first side portion 24, a second side portion 25, and an anesthesia screen 26. The main sheet 22 is flanked on either side by the first and second side portions 24 and 25. The top of main sheet 22 is connected to the anesthesia screen 26 to form a generally T-shaped drape with the anesthesia screen 26 forming the top of the “T” and the main sheet 22 and the first and second side portions 24 and 25 forming the center of the “T,” as shown more fully in FIG. 2. As shown in FIG. 1, surgical drape 21 has been placed over a patient 27 lying recumbent on a surgical table. The surgical drape 21 is positioned such that the anesthesia screen 26 can be placed over a set of standards 28 located on either side of the operating table. The patient 27 lies below the surgical drape 21, and the anesthesia screen 26 is positioned on the standards 28 (one standard 28 is depicted) such that the head of patient 27 is located behind the anesthesia screen 26 to obscure the patient's view of the surgical area. In this manner, the surgeon can isolate the surgical area from the rest of the patient's body to maintain the surgical area in a sterile state and the surgeon can obscure the patient's view of the surgical procedure. If desired, the anesthesia screen 26 may be selectively lowered so that the patient 27 can view the surgical area.

The surgical drape 21 may be constructed of any suitable material. As shown, the main sheet 22 and the anesthesia screen 26 of the surgical drape 21 are constructed of a multi-layer combination of spunbond and meltblown materials, commonly referred to as SMS non-woven fabric. This combination provides a lightweight, durable, repellent, and flexible fluid barrier between the patient and the sterile filed that is easily drapeable and soft to the touch. The first and second side portions 24 and 25 are made of a flexible fluid impervious and nonabsorbent material, such as polyethylene film, or may be SMS as well.

Referring now to FIG. 2, the main sheet 22 of the surgical drape 21 has a drape window 30. As shown, drape window 30 is a square-shaped opening in an area of main sheet 22 that may be positioned over the abdomen of a patient for a C-section procedure. The drape window 30 exposes the patient's skin and the area of the patient where the surgery will be performed so that the surgery can be performed through the drape window 30 of the main sheet 22. In this manner, the surgeon has access to the patient and the specific surgical area on which the procedure is to be performed while maintaining the sterile state of the surgical area.

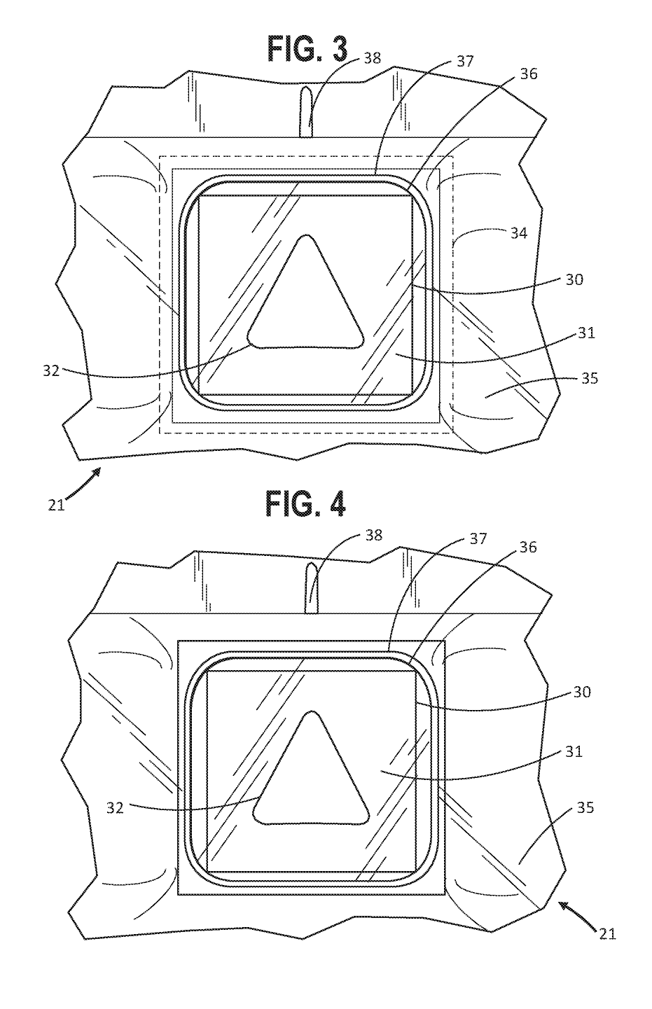

As shown, the drape window 30 is partially covered by an incise film 31. The incise film 31, which may be formed from a polyurethane or other suitable material, is a substantially transparent film. Notably, in many embodiments the purpose of the incise film will be to provide adhesion and a fluid seal proximal the abdominal incision, Although the term “incise film” will be used, in these embodiments it is not necessarily intended for the surgeon to make an incision through the film. In any case, the incise film 31 has a patient-facing side which includes an adhesive field suitable for adhering the incise film 31 to the skin of the patient in an area where the surgical procedure is to be performed. Before the drape is to be used in a surgical procedure and during packaging and storage, the adhesive field on the patient-facing side of the incise film 31 is covered by a removable backing 34 (shown further in FIG. 3). The removable backing 34 prevents the adhesive field on the incise film 31 from inadvertently sticking to other portions of the drape or to the patient's body before the drape is properly positioned over the patient 27. When the drape is to be placed over a patient, the removable backing 34 may be peeled away from the adhesive field on the patient-facing side of the incise film 31 to expose the adhesive field.

In the illustrated embodiment, the incise film 31 includes a generally triangular-shaped drape fenestration 32 which defines the fenestration in the drape. The drape fenestration 32 is an open void in the incise film 31 that permits surgical access to a region of a patient on which a surgical incision is to be made. In other embodiments, the drape fenestration 32 may take any suitable shape and size that would facilitate providing surgical access to a region of a patient adequate for the specific surgical procedure that is to be performed. Alternatively, the drape window of the main sheet may not be covered with any incise film, or may be fully covered with incise film (not shown). It is contemplated that in embodiments wherein the drape window does not include incise film, the drape window will itself define the fenestration in the drape.

As shown, the surgical drape 21 includes a fluid collection pouch 35 that substantially surrounds the drape window 30 of the main sheet 22. The fluid collection pouch 35 is made of a plastic material that is impervious to fluid. The fluid collection pouch 35 surrounds the drape window 30 in a sealing fashion such that any fluids released from the surgical site during the procedure and flowing over the drape will run off the main sheet 22 and into the fluid collection pouch 35. This prevents fluids from running off the main sheet 22 and migrating through the drape window 30 and onto the patient or the surgical bed or the floor of the operating room, where fluids are not desired. The fluid collection pouch 35 may include one or more suction ports 40 for connection to suction equipment for aspirating the fluids from the fluid collection pouch 35. Because the region surrounding the drape fenestration 32 is adhesively secured to the patient, fluids are inhibited from escaping beneath the perimeter of the drape fenestration.

With reference to FIG. 3, the fluid collection pouch 35 includes an opening 36 through which the surgeon may access the surgical area of the patient. The edges of the opening 36 may be bounded by a formable material 37, such as a malleable wire encased in plastic and formed to be integral to and generally coextensive with the edges of the opening 36. The formable material 37 allows the surgeon to shape the opening of the fluid collection pouch 35 to allow for easier access to the surgical area or to reconfigure the shape of the fluid collection pouch 35 in a manner that is most desirable for the particular procedure.

With reference again to FIG. 2, the main sheet 22 includes one or more line anchors 38 for securing wires or lines to the surgical drape 21. The line anchors 38 are composed of first and second strips of hook and loop material. The first strip is bound to the surgeon-facing side of the main sheet 22. The second strip is permanently bound at an end to the first strip. When the two strips are pressed together the hook and loop material disposed on opposing faces of the strips connect in a removable fashion. When the two strips are pulled apart, they separate at their ends opposite the ends permanently bound to one another. When the strips are separated, a tube or line may be placed between the two plies. When the two plies are joined, the hook and loop material captures the hose or line between the two plies of material. In this manner, tubes and lines used during a surgical procedure may be temporarily attached to the surgical drape 21 for purposes of organizing and routing those lines and wires.

The main sheet 22 also includes an optional absorbent pad 41, (shown in FIG. 2) which, as shown, is configured as a separate pad disposed on the main sheet 22. The absorbent pad 41 is located near the drape window 30 and near the fluid collection pouch 35. The absorbent pad 41 is made up of a material suitable for absorbing fluids generated during the surgical procedure. The absorbent pad 41 provides another measure of fluid retention in the instance where fluids are not collected by the fluid collection pouch 35.

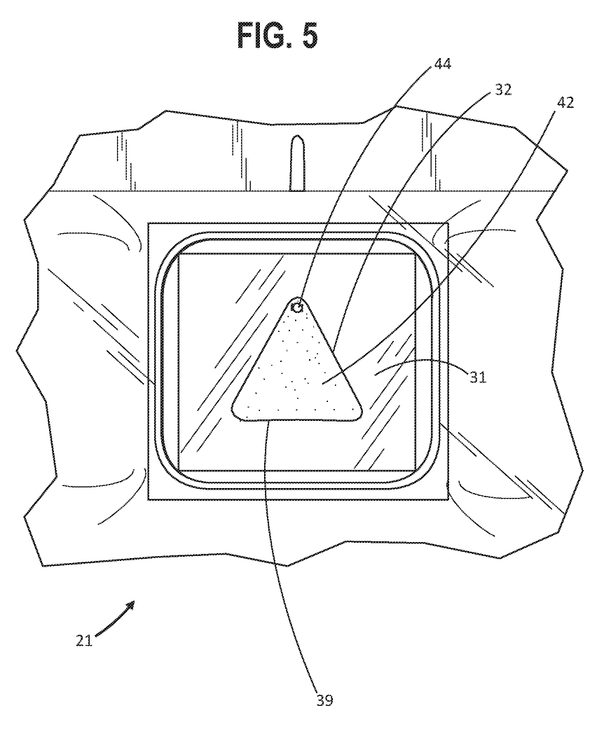

Referring now to FIG. 4, because the removable backing 34 has been removed from the patient-facing side of the incise film 31, the drape is now prepared to be positioned over a patient. Subsequently, as shown in FIG. 5, the incise film 31 has now been adhered to the patient's abdominal skin 42, which is visible through the drape fenestration 32. Desirably, to assist in orienting the surgical incision, the drape fenestration is sized such that the patient's navel 44 is disposed at the apex of the triangular fenestration 32 when the base of the fenestration 39 is disposed proximal the area to be incised. It is seen that the bib in the illustrated embodiment completely covers the drape fenestration when so positioned.

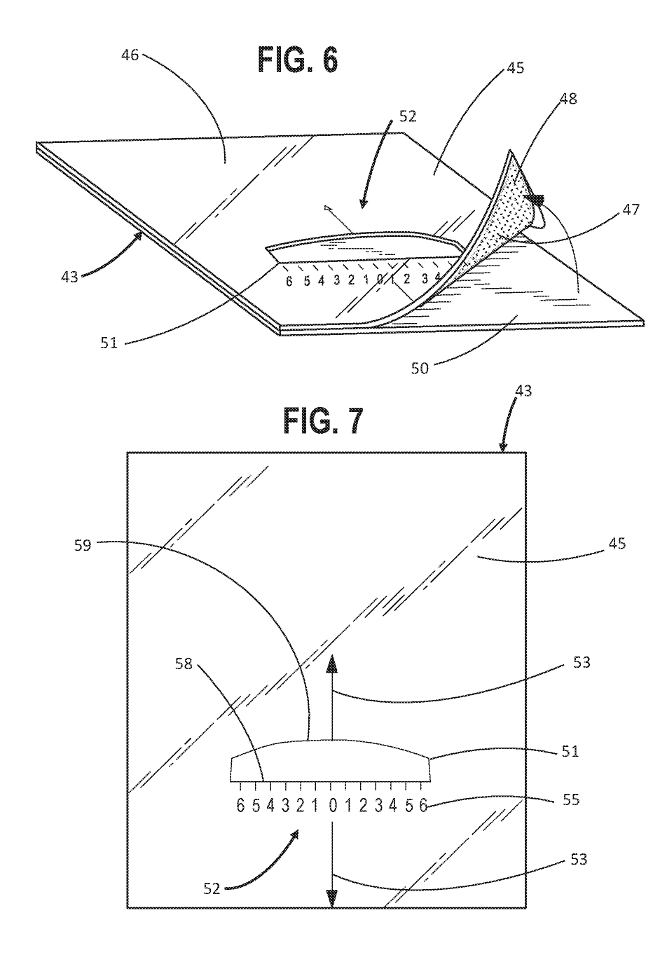

Referring to FIGS. 6 and 7, a surgical bib 43 that is usable with the surgical drape 21 (not shown in FIG. 6 or 7) is provided. The surgical bib 43 includes a flexible bib sheet 45 that may be constructed of any suitable material, and as shown is constructed of a flexible sheet of polypropylene film. It is contemplated that the bib should generally be composed of a material that is more durable than the incise film 31. This flexible bib sheet 45 has a surgeon-facing side 46 and a patient-facing side 47. The patient-facing side 47 includes an adhesive field 48. The adhesive field 48 comprises a coating of an adhesive material suitable for adhering the patient-facing side 47 of the flexible bib sheet 45 to a patient's skin in the region of the patient's body where a surgical procedure is to be performed. The adhesive field 48 of the patient-facing side 47 of the flexible bib sheet 45 may be covered with a removable bib backing 50. The removable bib backing 50 covers the adhesive field 48 before it is desired to adhere the flexible bib sheet 45 to a patient's skin. The removable bib backing 50 may be removed to expose the adhesive field 48 of the patient-facing side 47 of the flexible bib sheet 45 when it is desired to adhere the flexible bib sheet 45 to a patient's skin at the time of a surgical procedure. In alternative embodiments (not shown), the bib is manufactured without adhesive and the surgeon optionally applies adhesive to the bib or patient before positioning the bib. The flexible bib sheet 45 includes a bib fenestration 51. As shown, the bib fenestration 51 defines an area bound by a straight bottom edge 58 and an arced top edge 59. The bib fenestration 51 in other embodiments may take any suitable shape as may be desired for a C-section procedure or as may be designed for a particular surgical procedure. As shown, the bib and drape are manufactured to be a separate components, but it is contemplated in some embodiments (not shown) the bib may be manufactured as a physically attached component of the drape. In such cases the bib should be movable relative to the drape window to permit the bib to be positioned on the patient after the patient has been covered with the drape.

As shown, the flexible bib sheet 45 includes one or more surgical guide markings 52. The surgical guide markings 52 are printed in an area proximate to the bib fenestration 51. The surgical guide markings 52 are visible from the surgeon-facing side 46 of the flexible bib sheet 45 and to the extent that they are composed of letters or numerals are oriented to be read normally from the surgeon-facing side. The surgical guide markings 52 provide reference points to assist in locating and orienting the incision. With reference to FIG. 7, the guide markings 52 may include one or more ordinate indicia 53 that are positioned coincident to the central vertical axis of the flexible bib sheet 45 and that may be aligned with the patient's centerline. The surgical guide markings 52 may also include a bilateral measurement scale 55 of measurement units (e.g., centimeters) or other equally spaced markings that may be used to size an incision on either side of the ordinate indicia 53 to assist the surgeon in making a bilaterally symmetric incision about the patient's midline. The straight bottom edge 58 may serve as a lateral incision guide that provides a substantially straight edge to assist the surgeon in making a neat incision. Alternatively, the bid may be provided with a linear marking (not shown) printed on flexible bib sheet 45.

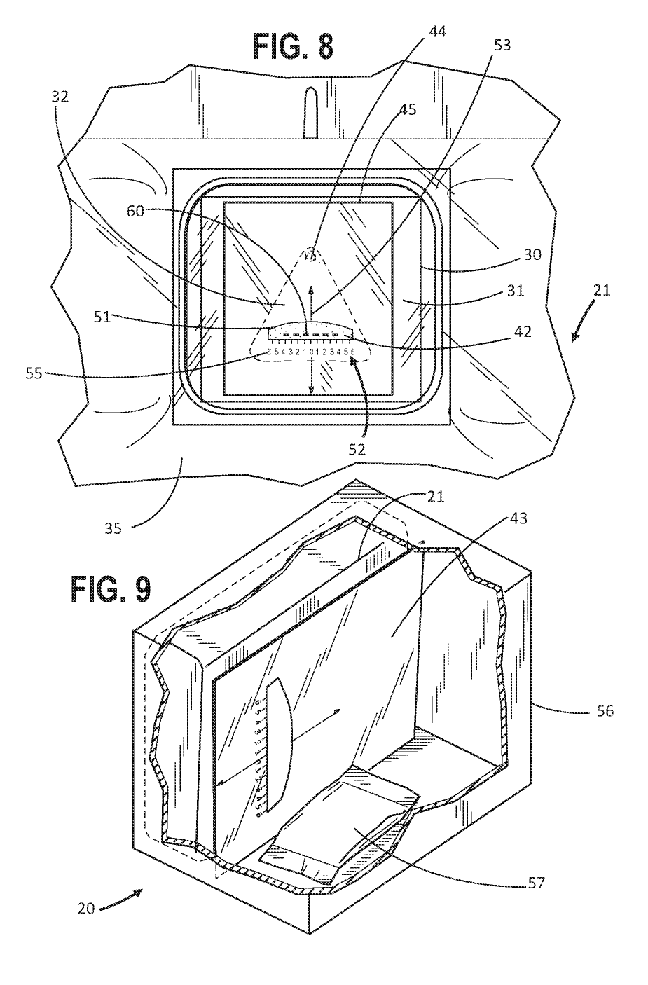

Referring to FIG. 8, the surgical bib 43 is sized to cover the drape fenestration 32. In embodiments where the drape window does not include incise film (not shown), the bib should be sized to at least partially cover the drape window and preferably substantially or completely cover the drape window. As illustrated, the removable bib backing 50 (not shown) has been removed from the patient-facing side of the flexible bib sheet 45, thereby exposing the adhesive field of the flexible bib sheet 45. The flexible bib sheet 45 is placed over the drape fenestration 32 and adhered partially to the patient and partially to incise film 31 covering the drape window 30 of the surgical drape 21. In this manner, the flexible bib sheet 45 provides a substantially leak proof seal within the drape fenestration 32 such that fluids cannot readily migrate between the flexible bib sheet 45 and the incise film 31 and otherwise are inhibited from escaping beneath the perimeter of the bib fenestration. In embodiments in which the drape fenestration is not bound by incise film, the flexible bib sheet 45 may be adhered directly to the drape.

In placing the flexible bib sheet 45 over the patient, the flexible bib sheet 45 is positioned over the drape fenestration 32 such that the bib fenestration 51 of the flexible bib sheet 45 is unobstructed by any portion of the incise film 31 and such that the bib fenestration 51 exposes an area of the patient's abdominal skin 42 at which the surgeon wishes to make an incision 60 (shown as a phantom line in FIG. 8). In this manner, the patient's abdominal skin 42 is exposed through the bib fenestration 51 and the incision 60 may be made unobstructed by any portion of the surgical drape 21 or flexible bib sheet 45.

The surgeon may use the ordinate indicia 53 to align the flexible bib sheet 45 to the patient's anatomy before it is adhered to the patient. As shown, the ordinate indicia 53 include upper and lower arrows. The arrow pointing towards the patient's navel 44 may be aligned with the patient's navel 44. The arrow pointing away from the patient's navel 44 may be aligned with the patient's vagina. In this manner, the surgical bib may be positioned and adhered to the patient in a way that aligns the flexible bib sheet 45 along the centerline of the patient with the ordinate indicia 53 generally corresponding to the patient's centerline and lateral incision guide 54.

Because the flexible bib sheet 45 is substantially transparent, when it is positioned over the patient, the patient's anatomy may be viewable through the flexible bib sheet 45. For example, the patient's navel 44 is viewable through the flexible bib sheet 45. This allows the surgeon to position the flexible bib sheet 45 using surgical guide markings 52 with reference to the patient's anatomy visible through the flexible bib sheet 45.

Referring to FIG. 9, the surgical drape kit 20 is shown in a package 56. Package 56 contains the surgical drape 21, the surgical bib 43, and a container 57 of adhesive. The surgical drape 21 may be wrapped inside the package in a sterile wrapping sheet (not shown). The container 57 may be used to provide adhesive material to the patient-facing side 47 of the flexible bib sheet 45 of the surgical bib 43. In one embodiment, the patient-facing side 47 of the flexible bib sheet 45 may not be provided with any adhesive material. In such an embodiment, the adhesive 57 may be applied to the patient-facing side 47 of the flexible bib sheet 45 at the time it is to be applied to a patient. The adhesive 57 adheres the flexible bib sheet 45 to the patient's abdominal skin 42 as well as the area of the surgical drape 21 surrounding the drape fenestration 32.

In another embodiment in which the patient-facing side 47 of the flexible bib sheet 45 is provided with adhesive field, adhesive 57 provided with surgical drape kit 20 may be used to supplement the adhesive properties of the adhesive field on the patient-facing side 47 of the flexible bib sheet 45. Alternatively, adhesive 57 may be applied to patient-facing side 47 of the flexible bib sheet 45 to restore its adhesive properties if, for example, the flexible bib sheet 45 must be removed and re-adhered to the patient's skin. Often when adhesive materials are removed from a patient's skin, they do not readily re-adhere to the patient's skin due to degradation of the adhesive or due to contamination of the adhesive field with other materials or fluids. Adhesive 57 may be used to restore or supplement adhesive properties of the flexible bib sheet 45 if it must be removed and repositioned. As shown, the bib and drape are physically separate components, although in some surgical applications it is contemplated that the bib may be pre-fastened to the drape.

In using the surgical drape kit 20 in a C-section procedure, the patient is first positioned on a surgical table and the surgical drape 21 is removed from the package 56. At this point, the surgical drape 21 may be enclosed within a secondary sterile wrapper (not shown). A member of the surgical team may remove the wrapped surgical drape 21 from the container, and, by using methods known in the field, may unwrap the surgical drape 21 and present it to a member of the surgical team that has performed decontamination, such that the sterile state of the surgical drape 21 may be maintained during setup of the surgical procedure. The drape and bib are placed as discussed above, and the surgeon may apply adhesive (or supplemental adhesive) to the bib. The bib may be used to assist in orienting and positioning the drape. In embodiments where the bib is pre-fastened, the bib and drape are placed at the same time. It is contemplated in such embodiments that the bib may be partially movable with respect to the drape to aid in orienting an incision. In any case, as heretofore indicated, the indicia may be used to place the incision and the straight edge 58 of the bib fenestration may be used to assist the surgeon in making a smooth, linear incision. Desirably, there is no incise film between the surgeon and the area of the abdomen immediately beneath the bib fenestration, such that there will be no bunching of the skin as might result in a jagged scar. The scale may assist the surgeon in achieving bilateral symmetry.

Via the adhesive connection of the bib to the patient surrounding the bib fenestration, and also because the drape incise film is adhesively secured to the patient, fluids released during the procedure will be inhibited from escaping beneath the drape. Instead, the fluids will be guided away over the bib and incise film into the fluid-collection pouch. The bib is preferably but not necessarily sized to completely cover the drape fenestration when in use. In such configurations, fluid will flow over the bib in areas above the drape fenestration, providing an additional safeguard against escaping fluids.

It is thus seen that the bib and kit are useful in assisting the surgeon in making a C-section incision are provided. It is contemplated that the procedure will assist the surgeon in leaving a scar that is neat and not jagged as a result of bunched incise film. Additionally, the bib assists in placing and orienting the incision such that the incision and resulting scar are bilaterally symmetric with respect to the centerline of the patient, and oriented generally perpendicular to the centerline rather than being skewed. Additionally, the bib and kit in some embodiments provide the heretofore described advantages of not requiring the incision to be made through incise film. The foregoing has been described with particular regard to C-section procedures, but the bib and kit are contemplated to find utility in other surgical applications.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A surgical bib comprising:
a flexible bib sheet comprising a sheet of flexible material having patient-facing side and a surgeon-facing side opposite the patient-facing side;
a bib fenestration formed in the flexible bib sheet for permitting surgical access to a patient through the bib fenestration;
at least one surgical guide marking on the flexible bib sheet visible on the surgeon-facing side of the flexible bib sheet; and
an adhesive field covering at least a portion of the patient-facing side of the flexible bib sheet and surrounding the bib fenestration, the adhesive field comprising an adhesive suitable for adhering at least a portion of the patient-facing side of the flexible bib sheet surrounding the bib fenestration to a surface of a patient's skin proximal a surgical area of the patient.

2. The surgical bib of claim 1, the at least one surgical guide marking comprising indicia for aligning the surgical bib to at least one anatomical feature of the patient.

3. The surgical bib of claim 1, the bib having a vertical axis, the at least one surgical guide marking comprising ordinate indicia for aligning the vertical axis to the navel and vagina of a patient.

4. The surgical bib of claim 3, the at least one surgical guide marking further comprising a bilateral measurement scale for guiding a surgeon in making an incision in the patient that is bilaterally symmetric about said vertical axis.

5. The surgical bib of claim 4, the flexible bib sheet being movable to a substantially flat configuration, the flexible bib sheet comprising a lateral incision guide that is generally perpendicular to said vertical axis when said flexible bib sheet is in said substantially flat configuration.

6. A surgical drape kit comprising:
a surgical drape comprising a drape material and having a drape fenestration; and
a flexible bib sheet, said flexible bib sheet comprising
a sheet of flexible material having a patient-facing side and a surgeon-facing side opposite the patient-facing side;
a bib fenestration formed in the flexible bib sheet for permitting surgical access to a patient through the bib fenestration;
at least one surgical guide marking on the flexible bib sheet visible on the surgeon-facing side of the flexible bib sheet; and
said bib being positionable over said drape fenestration and being of a size sufficient to at least partially cover said drape fenestration.

7. The surgical drape kit of claim 6, said bib comprising an adhesive field covering at least a portion of the patient-facing side of the flexible bib sheet and surrounding the bib fenestration, the adhesive field comprising an adhesive suitable for adhering at least a portion of the patient-facing side of the flexible bib sheet surrounding the bib fenestration to a surface of a patient's skin proximal a surgical area of the patient.

8. The surgical drape kit of claim 6, further comprising a container of an adhesive for applying adhesive to the flexible bib sheet, said adhesive an adhesive suitable for adhering at least a portion of the patient-facing side of the flexible bib sheet surrounding the bib fenestration to a surface of a patient's skin proximal a surgical area of the patient.

9. The surgical drape kit of claim 7, further comprising an adhesive container for supplying additional adhesive for the flexible bib sheet.

10. The surgical drape kit of claim 7, the drape including a drape window having an incise film disposed thereover, said drape fenestration comprising an aperture in the incise film, the incise film comprising a patient-facing side and an opposing surgeon-facing side and an adhesive field for adhering at least a portion of the patient-facing side of the incise film to a surface of a patient's skin proximal a surgical area of the patient.

11. The surgical drape kit of claim 7, the at least one surgical guide marking comprising indicia for aligning the surgical bib to at least one anatomical feature of the patient.

12. The surgical drape kit of claim 7, the bib having a vertical axis, the at least one surgical guide marking comprising ordinate indicia for aligning the vertical axis to the navel and vagina of a patient.

13. The surgical drape kit of claim 12, the at least one surgical guide marking further comprising a bilateral measurement scale for guiding a surgeon in making an incision in the patient that is bilaterally symmetric about said vertical axis.

14. The surgical drape kit of claim 13, the flexible bib sheet being movable to a substantially flat configuration, the flexible bib sheet comprising a lateral incision guide that is generally perpendicular to said vertical axis when said flexible bib sheet is in said substantially flat configuration.

15. The surgical drape kit of claim 14, the drape including a drape window having an incise film disposed thereover, said drape fenestration comprising an aperture in the incise film, the incise film comprising a patient-facing side and an opposing surgeon-facing side and an adhesive field for adhering at least a portion of the patient-facing side of the incise film to a surface of a patient's skin proximal a surgical area of the patient.

16. A method of performing surgery, comprising:
applying a surgical drape to a patient, the surgical drape including a drape fenestration;
positioning the surgical drape with respect to the patient to expose the surgical area of the patient through the drape fenestration;
positioning a flexible bib over the drape fenestration, the flexible bib including a bib fenestration and at least one surgical guide marking, said bib being of a size sufficient to at least partially cover said drape fenestration;
aligning the flexible bib over the surgical area of the patient by aligning the at least one surgical guide marking with at least one anatomical feature of the patient and aligning the bib fenestration over the surgical area to expose the surgical area through the bib fenestration;
adhering the aligned flexible bib to the surgical area of the patient and to the drape at a perimeter portion of the drape fenestration.

17. A method according to claim 16, said surgery comprising a caesarian section.

18. A method according to claim 17, said bib comprising a sheet of flexible material having a patient-facing side and a surgeon-facing side opposite the patient-facing side and an adhesive field covering at least a portion of the patient-facing side of the flexible bib sheet and surrounding the bib fenestration, the adhesive field comprising an adhesive suitable for adhering at least a portion of the patient-facing side of the flexible bib sheet surrounding the bib fenestration to a surface of a patient's skin proximal a surgical area of the patient.

19. A method according to claim 17, the drape including a drape window having an incise film disposed thereover, said drape fenestration comprising an aperture in the incise film, the incise film comprising a patient-facing side and an opposing surgeon-facing side and an adhesive field for adhering at least a portion of the patient-facing side of the incise film to a surface of a patient's skin proximal a surgical area of the patient.

20. A method according to claim 17, the bib having a vertical axis, the at least one surgical guide marking comprising ordinate indicia for aligning the vertical axis to the navel and vagina of a patient, the method comprising aligning said bib in accordance with said ordinate indicia.

21. A method according to claim 17, the at least one surgical guide marking further comprising a bilateral measurement scale for guiding a surgeon in making an incision in the patient that is bilaterally symmetric about said vertical axis, the method including making an incision using said bilateral measurement scale as a guide.

22. A method according to claim 17, the flexible bib sheet being movable to a substantially flat configuration, the flexible bib sheet comprising a lateral incision guide that is generally perpendicular to said vertical axis when said flexible bib sheet is in said substantially flat configuration, the method comprising making said incision using said lateral incision guide as a guide.

23. A method according to claim 22, the drape including a drape window having an incise film disposed thereover, said drape fenestration comprising an aperture in the incise film, the incise film comprising a patient-facing side and an opposing surgeon-facing side and an adhesive field for adhering at least a portion of the patient-facing side of the incise film to a surface of a patient's skin proximal a surgical area of the patient.
